(19) United States
(12) Patent Application Publication
(10) Pub. No.: US 2005/0032771 A1
(43) Pub. Date: Feb. 10, 2005

(54) Process for the preparation of cefixime via alkyl-or aryl-sulfonates

(76) Inventors: Walter Cabri, Rozzano (IT); Marco Alpegiani, Milano (IT); Giovanni Pozzi, Besana Brianza (IT); Gomez Patricio Martin, Salamanca (ES); Francesco Oliva, Milano (IT)

Correspondence Address: ROTHWELL, FIGG, ERNST & MANBECK, P.C., 1425 K STREET, N.W., SUITE 800, WASHINGTON, DC, 20005, US

(21) Appl. No.: 10/494,700

(22) PCT Filed: Oct. 11, 2002
(86) PCT No.: PCT/EP02/11405

(30) Foreign Application Priority Data
Nov. 9, 2001 (IT) MI200IA002364

(51) Int. Cl. A61K 31/545; C07D 501/14

(52) U.S. Cl. 514/202; 540/222

(57) ABSTRACT

A process for the preparation Cefixime, namely 7-[2-(2-aminothiazol-4-yl)-2-(carboxymethoxyimino)acetamido]-3-vinyl-3-cephem-4-carboxylic acid, via alkyl- or aryl- sulfonates, of general formula (IA).

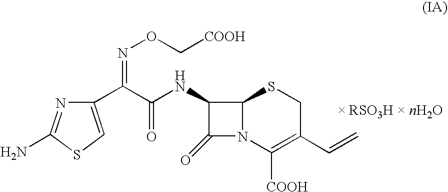

The present invention relates to a process for the preparation of Cefixime via crystalline alkyl- or aryl- sulfonates of general formula (IA), 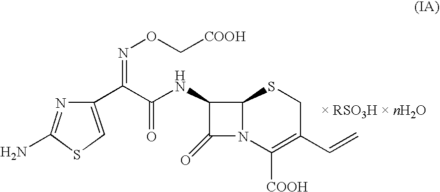
which can be obtained starting from a 7-amino-3-vinyl-3-cephem-4-carboxylic acid derivative of general formula (II) without recovering any intermediate.

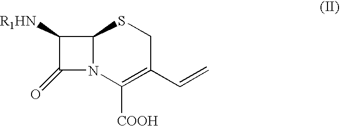

### PRIOR ART

Cefixime (I), whose chemical name is [6R-(6α,7β(Z)]-7-{[(2-amino-4-thiazole)[(carboxymethoxy)imino]acetyl]amino}-3-ethenyl-8-oxo-5-thia-1-azabicyclo[4.2.0]oct-2-ene-2-carboxylic acid, is a semisynthetic cephalosporin for the oral use, which exerts its antibiotic action by inhibiting the synthesis of the bacterium cell wall. This antibiotic is highly stable to beta-lactamases: as a consequence, it is active against a number of organisms resistant to penicillins and to some cephalosporins, inter alia Streptococcus pneumoniae, Haemophilus influenzae, Escherichia coli and Neisseria gonorrhoeae.

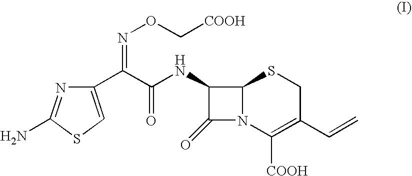

Based on the knowledge from literature, Cefixime can be obtained through different procedures, for example according to Scheme 1, wherein compounds of formula (II), (III) and (IV) also include any salts or solvates thereof.

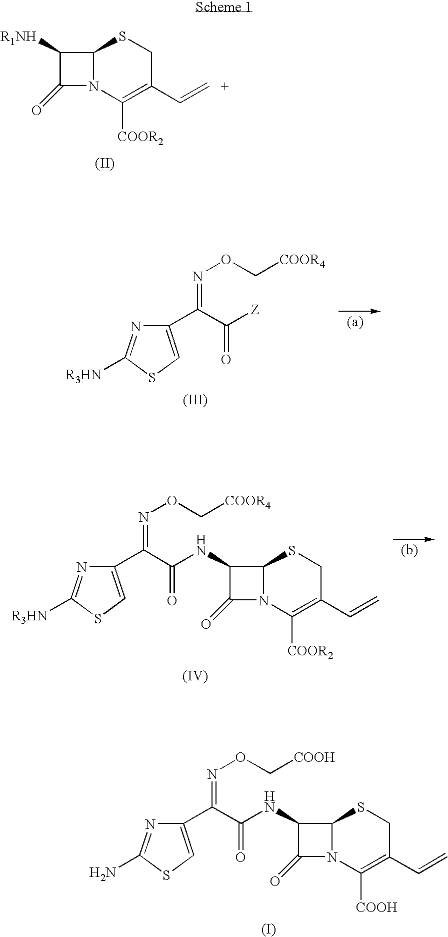

A 7-amino-3-vinyl-3-cephem-4-carboxylic acid derivative of formula (II), wherein
R₁ is hydrogen or a silyl group, preferably trimethylsilyl,
R₂ is hydrogen or a carboxy-protecting group, for example a silyl group, preferably trimethylsilyl, a C₁₋₆ straight or branched alkyl group, a benzyl, benzhydryl or trityl group wherein each benzene ring can be unsubstituted or substituted with one or more methoxy, nitro and/or methyl groups,
is reacted with a 2-(aminothiazol-4-yl)-2-(carboxymethoxyimino)acetic acid derivative of formula (III), wherein
R₃ is hydrogen or an amino-protecting group, for example a C₁₋₆ straight or branched acyl group, an alkyl - or aryl- oxycarbonyl group, or a trityl group wherein each benzene ring is unsubstituted or substituted with one or more methoxy, nitro and/or methyl groups,
R₄ is a carboxy-protecting group, for example a C₁₋₆ straight or branched alkyl group, a benzyl, benzhydryl o trityl group wherein each benzene ring is unsubstituted or substituted with one or more methoxy, nitro and/or methyl groups,
Z is a carboxy-activating agent which, together with the carbonyl group to which is bound, forms an acid chloride, an organic or inorganic acid anhydride, an ester, a thioester or an amide,
to give a 7-[2-(2-aminothiazol-4-yl)-2-(carboxymethoxyimino)acetamido]-3-vinyl-3-cephem-4-carboxylic acid derivative of formula (IV), wherein R₂, R₃ and R₄ have the meanings defined above, for example 7-[2-(2-aminothiazol-4-yl)-2-(tert-butoxycarbonylmethoxyimino)acetamido]-3-vinyl-3-cephem-4-carboxylic acid of formula (IVA), 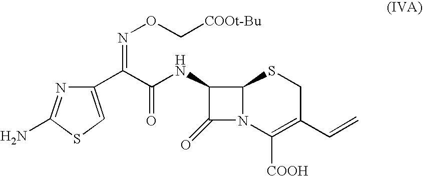
   from which the R₂, R₃ and R₄ groups, which are different from hydrogen, are subsequently removed to obtain Cefixime (I).

The disadvantage of this synthetic process is that intermediate (IV) has to be isolated.

Typically, derivatives of general formula (IV), such as compound (IVA), are obtained as amorphous solids and, due to their high solubility in most organic solvents, they have to be isolated by using solvents, such as ethers, which however also promote the precipitation of undesired by-products (EP 0 306 30). Derivatives (IV) in which R₂ is hydrogen can also be isolated from water in the form of free acids, but they are very difficult to filter and dry (WO 98/31685).

Compounds (IV) can also be isolated in the form of amine salts from organic solvents or mixtures thereof (WO 98/31685 and WO 99/51607). This method, however, provides lower yields than the above mentioned ones.

Compounds (IV) can be transformed into Cefixime (I) by removing the protective groups under acid or basic conditions.

Removal of the protective groups in basic conditions provides low yields due to the high instability of the cephalosporin in said conditions (DE 19846449).

Typically, the protective groups defined above are removed under acid conditions, although also in this case high quality compounds are hardly obtained in good yields.

For example, removal of the protective groups, such as benzhydryl or tert-butyl, using trifluoroacetic acid or aluminium trichloride in anisole, hydrochloric acid or methanesulfonic or para-toluenesulfonic acids in suitable solvents, yields low purity amorphous Cefixime salts, which have to undergo further purification steps before the conversion into Cefixime (U.S. Pat. No. 4,409,214, WO 95/33753, EP 0 30 630 and WO 98/06723).

The use of sulfuric acid during the deprotection reaction, under the conditions disclosed in WO 98/31685, induces precipitation of Cefixime sulfate, which however is not obtained in high purity due to the poor solubility of the by-products sulfate salts. It has moreover been observed that water present in the reaction medium, in part deriving from the sulfuric acid used, decreases the reaction rate, prevents the complete conversion of the starting product, favors degradation of the cephalosporin while preventing aggregation of Cefixime sulfate during the precipitation, which negatively affects filtration and isolation of the product.

For these reasons, the use of sulfuric acid in a process in which derivatives (IV) are not isolated, does not yield Cefixime sulfate of suitable purity for transformation into Cefixime (I).

It now has been found, and this is the object of the invention, that, without isolating intermediates (IV), but treating them directly with alkyl- or aryl- sulfonic acids, the respective highly pure Cefixime crystalline salts can be obtained in good yield, by means of selective crystallization of the respective Cefixime alkyl- or aryl- sulfonates.

The process of the invention for the preparation of Cefixime is therefore more convenient and free from the above described drawbacks.

### DETAILED DESCRIPTION OF THE INVENTION

The present invention relates to a process for the production of Cefixime (illustrated in Scheme 2), characterized by the treatment of compounds of general formula (IV) with alkyl- or aryl- sulfonic acids, to give Cefixime crystalline salts of formula (IA) and by the conversion of the latter into Cefixime, either anhydrous or hydrated, preferably trihydrate.

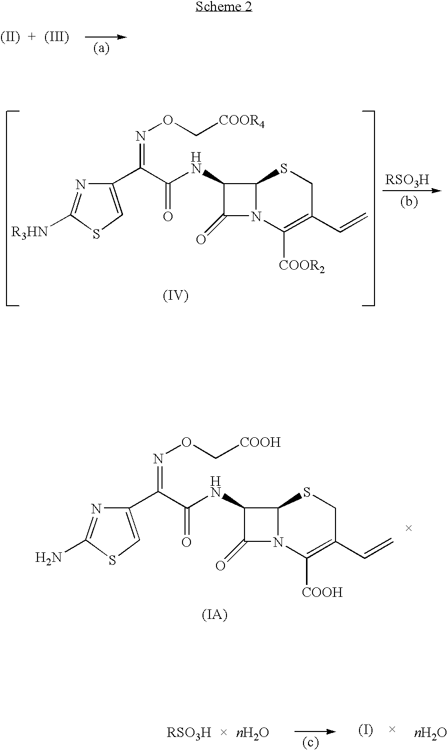

The process of the invention comprises:

(a) reacting a 7-amino-3-vinyl-3-cephem-4-carboxylic acid derivative of formula (II), as defined above,

with a 2-(aminothiazol-4-yl)-2-(carboxymethoxyimino)acetic acid reactive derivative of formula (III), as defined above,

to give a 7-[2-(aminothiazol-4-yl)-2-(carboxymethoxyimino)-acetamido]-3-vinyl-3-cephem-4-carboxylic acid derivative of formula (IV);

(b) directly reacting the crude compound of formula (IV) from step (a) with an alkyl- or aryl- sulfonic acid to give a Cefixime crystalline salt (IA);

(c) converting salts (IA) into Cefixime (I), which can be isolated as a highly pure solvate, for example trihydrate.

For the purposes of the present invention:

In compounds (II), R₁ is hydrogen or a silyl group, preferably trimethylsilyl; R₂ is hydrogen or a silyl, preferably trimethylsilyl, tert-butyl, p-methoxybenzyl, 3,4-dimethoxybenzyl, benzhydryl or bis(p-methoxyphenyl)methyl group.

In compounds (III), R₃ is hydrogen or a formyl, trityl, tert-butoxycarbonyl or p-methoxybenzyloxycarbonyl group; R₄ is a tert-butyl, p-methoxybenzyl, 3,4-dimethoxybenzyl, benzhydryl, bis(p-methoxyphenyl)methyl or trityl group and Z is a carboxy-activating group, selected from —Cl, —S-mercaptobenzothiazolyl, —O—P⁺(Ph)₃Cl⁻, —O—P(S)(OEt)₂, —O—P(O)(OEt)₂, —O—SO₂Me, —O—SO₂Ph, —O—SO₂-pTol, —O—COtBu, —O—C(O)OEt, —O-benzotriazol-1-yl, —S-(2-methyl-thiadiazol-5-yl), —O—CH═N⁺(CH₃)₂Cl⁻ or —benzotriazol-1-yl-3-oxide;

“Alkylsulfonic acids” means alkylsulfonic acids wherein R is a C₁-C₆ straight or branched alkyl.

“Arylsulfonic acids” means arylsulfonic acids wherein R is a benzene or naphthalene ring.

Both the alkyl and aryl groups can optionally have one or more substituents selected from: halogens, preferably fluorine, chlorine, bromine; hydroxy, carboxy, nitro, sulfo, methyl groups. Preferred alkyl sulfonic acids are methanesulfonic, ethanesulfonic, trifluoromethanesulfonic acids, more preferably methanesulfonic acid. Preferred arylsulfonic acids are benzenesulfonic, para-toluenesulfonic, mesitylenesulfonic acids.

In compounds (IV), R₂, R₃ and R₄ have the meanings described above.

Compounds (II), (III) and (IV) also include possible salts or solvates thereof.

In salts (IA), either anhydrous or hydrate, “n” ranges from 0 to 3 and is preferably 1; a particularly preferred compound of the invention is Cefixime methanesulfonate monohydrate.

According to a preferred embodiment of the invention, compound (II) is 7-amino-3-vinyl-3-cephem-4-carboxylic acid of formula (IIA), 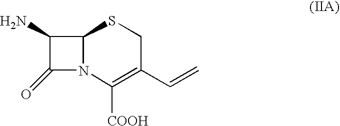
and compound (III) is 2-(aminothiazol-4-yl)-2-(tert-butoxycarbonylmethoxyimino)acetic acid S-mercaptobenzothiazole ester of formula (IIIA) 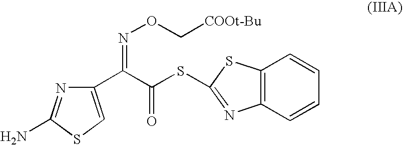
or 2-(aminothiazol-4-yl)-2-(tert-butoxycarbonylmethoxyimino)acetic acid O-diethylthiophosphoric ester of formula (IIIB),

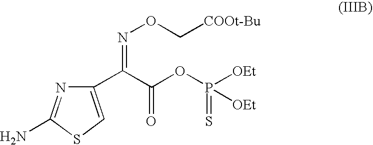

By way of example, the use of compounds (IIIA) or (IIIB) yields compound (IVA).

The reaction between compounds (II) and (III), described in step (a), can be carried out following procedures known in literature (J. Antibiotics (1985), 38(12), pages 1738-1751, WO 95/33753, EP 0 30 630, U.S. Pat. No. 5,003,073, WO 98/31685, WO 98/06723 and/or WO 99/51607).

For example, the salt of compound (IIA) with a tertiary amine, preferably triethylamine, N-methylmorpholine, N-ethyldiisopropylamine, or with an amidine, preferably 1,8-diazabicyclo[5.4.0]undec-7-ene (DBU), or with a guanidine, preferably tetramethyl guanidine, is reacted with the S-mercaptobenzothiazole ester (IIIA), optionally as solvate with N-methylpyrrolidinone, N,N-dimethylformamide or formamide; or with the O-diethylthiophosphoric ester (IIIB), in an organic solvent selected from a halogenated hydrocarbon such as dichloromethane, an ester such as ethyl acetate or methyl acetate, an ether such as tetrahydrofaran, or in mixtures of said solvents, optionally in the presence of a co-solvent, for example an alcohol such as methanol or ethanol, an amide such as N,N-dimethylformamide, or water, at a temperature ranging from −20° C. to +80° C., preferably from 0° C. to 40° C.

Step (b) can be carried out as follows:
a crude compound of formula (IV) from step (a), e.g. compound (IVA), can be transformed into a Cefixime salt (IA) by treatment with RSO₃H sulfonic acids as described above, in an organic solvent, for example an ester such as ethyl acetate, methyl acetate, ethyl formate or dimethylcarbonate, a ketone such as acetone, methyl ethyl ketone or methyl isobutyl ketone, a nitrile such as acetonitrile or propionitrile, an ether such as tetrahydrofuran, or other solvents such as methylene chloride or mixtures thereof, if desired in the presence of a co-solvent, for example an organic acid, such as formic, acetic or propionic acids.

The amounts of alkyl- or aryl- sulfonic acid can be stoichiometric to the compound of formula (IV) or in a molar excess up to 6 times, and is usually from 2 to 5 equivalents. The reaction temperature can range from −20° C. to 50° C., preferably from 0° C. to 40° C.

For carrying out step (b), the organic phase of the reaction mixture from step (a) can first be concentrated to small volume, for example from 10% to 60% of the starting volume. Concentration is typically effected by evaporation under reduced pressure at a temperature which can range from 0° C. to 60° C., preferably from 10° C. to 40° C. Alternatively, the solvent used in step (a) can be almost completely evaporated off and replaced with another solvent selected from those reported above, to the desired volume.

Cefixime alkyl- or aryl- sulfonates precipitate in the crystalline form from the reaction mixture and can easily be recovered by filtration or centrifugation. Crystallization of Cefixime salts (IA), during the removal of the protective groups, assists the complete transformation of the derivatives of formula (IV) while shielding Cefixime (I) from the aggressive conditions of the reaction medium, thus reducing degradation phenomena, with advantages in terms of yield and purity of the resulting Cefixime (IA) salts. Salts (IA) can be obtained either in the anhydrous or in the hydrate form. By way of example, starting from compound of formula (IVA) and using methanesulfonic acid, highly pure, crystalline Cefixime methanesulfonate monohydrate is obtained. Hydration water can be almost completely removed by drying under reduced pressure, thus improving the stability of the product. Typically, Cefixime methanesulfonate with a 0.5% water content or lower can be obtained after drying. Said salt, under normal humidity conditions, tends to rehydrate until stabilization to a water content corresponding to the one theoretically necessary for Cefixime methanesulfonate monohydrate.

Step (c) can be carried out according to any conventional method used in the synthesis of cephalosporins, for example by treatment with an organic base, such as a tertiary amine, or with an inorganic base, such as ammonia, alkali (for example sodium or potassium) carbonates, bicarbonates, hydroxides or phosphates, and subsequent treatment of the resulting salts with conventional acids. The reaction solvent can be water, or a mixture of water and alcohols, such as methanol, ethanol, propanol or butanol; ketones, such as acetone or methyl ethyl ketone, or other solvents such as tetrahydrofuran or acetonitrile.

Cefixime (I) can be obtained in the form of hydrate, preferably trihydrate, of other solvates, or unsolvated.

Cefixime (I) can also be obtained directly from the reaction mixture from step (b) by neutralization of the salt with conventional bases.

The following examples illustrate the invention in greater detail.

### EXAMPLE 1

### Preparation of Cefixime Methanesulfonate

To a suspension of 7-amino-3-vinyl-3-cephem-4-carboxylic acid (11.25 g), 2-(aminothiazol-4-yl)-2-(tert-butoxycarbonylmethoxyimino)acetic acid S-mercaptobenzothiazole ester (23.88 g) in ethyl acetate (EtOAc, 266 ml) and water (9 ml), cooled at 2° C., triethylamine (TEA, 13.9 ml) is added in 15 minutes and the mixture is kept under vigorous stirring at this temperature to obtain the complete conversion of 7-amino-3-vinyl-3-cephem-4-carboxylic acid (HPLC analysis). After completion of the reaction, water is added and pH is adjusted to 2.1 with diluted sulfuric acid. The phases are separated and the aqueous phase is re-extracted with EtOAc. Water is added to the combined organic extracts and pH is adjusted to 7.0 with aqueous sodium hydroxide. The phases are separated and the organic phase is re-extracted with water. The combined aqueous extracts are added with EtOAc (150 ml), and pH is adjusted to 2.1 with diluted sulfuric acid. The phases are separated, then the aqueous phase is re-extracted with EtOAc. The organic extracts are combined and concentrated to a volume of 120 ml, then acetonitrile (CH₃CN, 100 ml), formic acid (HCOOH, 22 ml), and methanesulfonic acid (MeSO₃H,13.2 ml) are added in succession, keeping the temperature between 30° and 35° C. After reacting for 1 hour (HPLC analysis), the mixture is cooled to 2° C., the precipitate is filtered, washed with acetonitrile and dried to obtain 20.86 g of Cefixime methanesulfonate.

¹H-NMR (D₂O, 300 MHz): 7.13 (1H, s, H-heteroaryl), 6.90 (1H, dd, J=17.7 and 11.4 Hz, —CH═CH₂), 5.72 (1H, d, J=4.8 Hz, —CONH—CH—CON), 5.52 (1H, d, J=17.4 Hz, —CH═CHH trans), 5.32 (1H, d, J=11.4 Hz, —CH═CHH cis), 5.17 (1H, d, J=4.8 Hz, —CON—CH—), 4.78 (2H, s), 3.69 (1H, AB system, J_{AB}=17.7 Hz), 3.57 (1H, AB system, J_{AB}=17.7 Hz), 2.69 (3H, s, CH₃SO₃—).

¹³C-NMR (D₂O, 300 MHz): 173.6, 170.9, 166.4, 164.6, 161.6, 144.0, 131.5, 130.2, 128.4, 125.1, 119.0, 112.5, 71.8, 59.2, 57.6, 38.6, 24.0.

### EXAMPLE 2

### Preparation of Cefixime Methanesulfonate

The procedure described in Example 1 is followed but, after concentrating the combined organic extracts to a volume of 220 ml, HCOOH (22 ml) and MeSO₃H (13.2 ml) are added in succession, keeping the temperature between 30° and 35° C. After reacting for 1 hour (HPLC analysis), the mixture is cooled to 2° C., the product is filtered, washed with ethyl acetate and dried to obtain 24.40 g of Cefixime methanesulfonate monohydrate.

### EXAMPLE 3

### Preparation of Cefixime Methanesulfonate

To a suspension of 7-amino-3-vinyl-3-cephem-4-carboxylic acid (11.25 g), 2-(aminothiazol-4-yl)-2-(tert-butoxycarbonylmethoxyimino)acetic acid S-mercaptobenzothiazole ester (24.18 g) in EtOAc (400 ml) and water (13.5 ml), TEA (13.5 ml) is added in 15 minutes, keeping the mixture at a temperature between 20° and 22° C. under vigorous stirring until obtaining the complete conversion of 7-amino-3-vinyl-3-cephem-4-carboxylic acid (HPLC analysis). After completion of the reaction, water is added and pH is adjusted to 2.1 with diluted sulfuric acid. The phases are separated and the aqueous phase is re-extracted with EtOAc. Water is added to the combined organic extracts and pH is adjusted to 7.0 with aqueous sodium hydroxide. The organic phase is separated and re-extracted with water. The combined aqueous extracts are added with EtOAc (150 ml) and pH is adjusted to 2.1 with sulfuric acid. The phases are separated, then the aqueous phase is re-extracted with EtOAc. The organic extracts are combined and concentrated to obtain an oil, which is taken up into CH₃CN (153 ml) and HCOOH (5.5 ml). MeSO₃H (13.1 ml) is added to this solution, keeping the temperature between 30° and 35° C. After reacting for 1 hour (HPLC analysis), the mixture is cooled to 2° C., the residue filtered, washed with acetonitrile and dried to obtain 23.11 g of Cefixime methanesulfonate monohydrate.

### EXAMPLE 4

### Preparation of Cefixime Methanesulfonate

To a suspension of 7-amino-3-vinyl-3-cephem-4-carboxylic acid (50.0 g), 2-(aminothiazol-4-yl)-2-(tert-butoxycarbonylmethoxyimino)acetic acid S-mercaptobenzothiazole ester (116.0 g) in EtOAc (1200 ml), water (45 ml) and methanol (115 ml), cooled at 2° C., TEA (13.5 ml) is added in 15 minutes and the mixture is kept under vigorous stirring at this temperature to obtain the complete conversion of 7-amino-3-vinyl-3-cephem-4-carboxylic acid (HPLC analysis). After completion of the reaction, water is added and pH is adjusted to 2.1 with aqueous hydrochloric acid. The aqueous phase is separated and re-extracted with EtOAc. Water is added to the combined organic extracts and pH is adjusted to 7.0 with aqueous sodium hydroxide. The organic phase is separated and re-extracted with water. The combined aqueous extracts are added with EtOAc and pH is adjusted to 2.1 with aqueous hydrochloric acid. The phases are separated and the aqueous phase is re-extracted with EtOAc. The combined organic extracts are concentrated to obtain an oil which is taken up into CH₃CN (890 ml) and HCOOH (31.8 ml). MeSO₃H (76.7 ml) is added to the resulting solution, keeping the temperature between 30° and 35° C. After reacting for 1 hour (HPLC analysis), the mixture is cooled to 2° C., the residue is filtered, washed with ethyl acetate and dried to obtain 124.2 g of Cefixime methanesulfonate monohydrate.

### EXAMPLE 5

### Preparation of Cefixime Methanesulfonate

To a solution of 7-[2-(2-aminothiazol-4-yl)-2-(tert-butoxycarbonylmethoxyimino) acetamido]-3 -vinyl-3 -cephem-4-carboxylic acid (10.0 g) in ethyl acetate (50 ml), acetone (20 ml) and formic acid (2.5 ml), methanesulfonic acid (6.0 ml) is added keeping the temperature between 30° and 35° C. After reacting for 1 hour (HPLC analysis), the mixture is cooled to 2° C., the residue is filtered, washed with ethyl acetate and dried to obtain 9.22 g of Cefixime methanesulfonate monohydrate.

1-13 (Cancelled)

14. A process for the preparation of Cefixime (I) 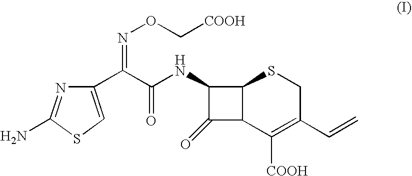
comprising the following steps:
a) reacting a 7-amino-3-vinyl-3-cephem-4-carboxylic acid derivative of formula (II) 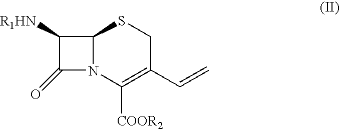
wherein
R₁ is hydrogen or a trimethylsilyl group;
R₂ is hydrogen or a trimethylsilyl, tert-butyl, p-methoxybenzyl, 3,4-dimethoxybenzyl, benzhydryl or bis(p-methoxyphenyl)methyl group;
with a 2-(aminothiazol-4-yl)-2-(carboxymethoxyimino) acetic acid derivative of formula (III) 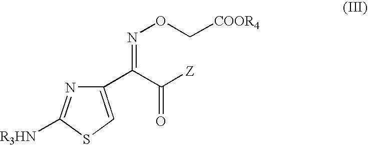
wherein
R₃ is hydrogen, a trityl, tert-butoxycarbonyl or p-methoxybenzyloxycarbonyl group;
R₄ is tert-butyl, p-methoxybenzyl, 3,4-dimethoxybenzyl, benzhydryl, bis(p-methoxyphenyl)methyl or trityl group;
Z is a carboxy-activating group selected from: —Cl, —S-mercaptobenzothiazolyl, —O—P⁺(Ph)₃Cl, —O—P(S)(OEt)₂, —O—P(O)(OEt)₂, —O—SO₂Me, —O—SO₂Ph, —O—SO₂-pTol, —O—COtBu, —O—C(O)OEt, —O-benzotriazol-1-yl, —S-(2-methyl-thiadiazol-5-yl), —O—CH═N⁺(CH₃)₂Cl⁻ or-benzotriazol-1-yl-3-oxide,
to give a 7-[2-(aminothiazol-4-yl)-2-(carboxymethoxyimino)acetamido]-3-vinyl-3-cephem-4-carboxylic acid derivative of formula (IV) 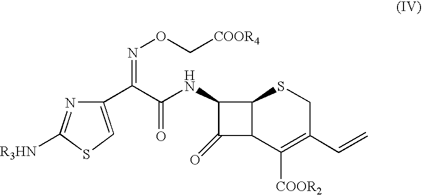
wherein R₂, R₃ and R₄ have the meanings defined above,
b) directly reacting a 7-[2-(aminothiazol-4-yl)-2-(carboxymethoxyimino)-acetamido]-3-vinyl-3-cephem-4-carboxylic acid derivative of formula (IV), from step a), with RSO₃H alkyl- or aryl- sulfonic acids to give crystalline salts (IA) 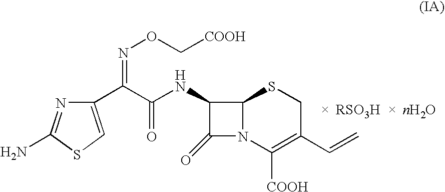
wherein
n is an integer ranging from 0 to 3,
R is a C₁-C₆ straight or branched chain; or a benzene or naphthalene ring optionally substituted with one or more substituents selected from fluorine, chlorine, bromine, hydroxyl, carboxy, nitro, sulfo, methyl groups,
c) converting crystalline salts (IA) into Cefixime (I).

15. A process as claimed in claim 14, wherein the alkylsulfonic acids are methanesulfonic, ethanesulfonic and trifluoromethanesulfonic acids.

16. A process as claimed in claim 14, wherein the alkylsufonic acid is methanesulfonic acid.

17. A process as claimed in claim 14, wherein the alkylsulfonic acids are benzenesulfonic, para-toluenesulfonic and mesitylenesulfonic acids.

18. A process as claimed in claim 14, wherein the compound of formula (II) is 7-amino-3-vinyl-3-cephem-4-carboxylic acid of formula (IIA)

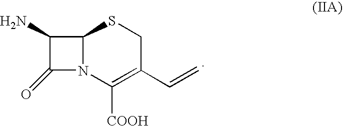

19. A process as claimed in claim 14, wherein the compound of formula (III) is 2-(aminothiazol-4-yl)-2-(tert-butoxycarbonylmethoxyimino)acetic acid S-mercaptobenzothiazole ester of formula (IIIA)

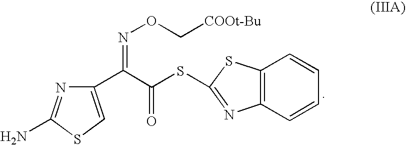

20. A process as claimed in claim 14, wherein the compound of formula (III) is 2-(aminothiazol-4-yl)-2-(tert-butoxycarbonyl-methoxyimino)acetic acid O-diethylthiophosphoric ester of formula (IIIB)

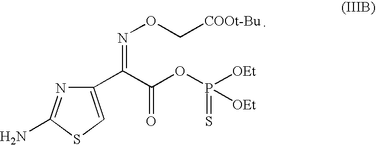

21. A process as claimed in claim 14, wherein Cefixime of formula (I) is obtained as the trihydrate.

22. Cefixime crystalline salts with alkyl- or arylsulfonic acids of formula (IA) 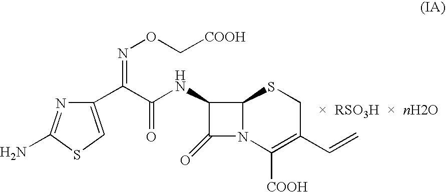
wherein
n is an integer ranging from 0 to 3,
R is a C₁-C₆ straight or branched chain; or a benzene or naphthalene ring.

23. Crystalline salts as claimed in claim 22, wherein R is selected from methyl, ethyl or trifluoromethyl.

24. Cystalline salts as claimed in claim 22, wherein R is selected from phenyl, p-tolyl or mesityl.

25. Cystalline salts as claimed in claim 22, wherein R has one or more substituent selected from fluorine, chlorine, bromine, hydroxy, carboxy, nitro, sulfo, methyl groups.

26. A crystalline salt as claimed in claim 23, wherein R is methyl and n is 1.
